(19) United States
(12) Patent Application Publication
(10) Pub. No.: US 2023/0190139 A1
(43) Pub. Date: Jun. 22, 2023

(54) SYSTEMS AND METHODS FOR IMAGE-BASED ANALYSIS OF ANATOMICAL FEATURES

(71) Applicant: Stryker Corporation, Kalamazoo, MI (US)

(72) Inventors: Ruth GODBEY, Sunnyvale, CA (US); Brian FOUTS, Morgan Hill, CA (US); Floor Mariet LAMBERS, Basel (CH)

(73) Assignee: Stryker Corporation, Kalamazoo, MI (US)

(21) Appl. No.: 18/069,976

(22) Filed: Dec. 21, 2022

Related U.S. Application Data
(60) Provisional application No. 63/292,412, filed on Dec. 21, 2021.

(51) Int. Cl. A61B 5/11; A61B 5/00

(52) U.S. Cl. CPC A61B 5/1128; A61B 5/1121; A61B 5/4571; A61B 5/7267; A61B 5/0082; A61B 2505/05; A61B 2576/02

(57) ABSTRACT

A method of generating a measurement of anatomy of interest from two-dimensional imaging includes receiving two-dimensional imaging associated with anatomy of interest; detecting a plurality of anatomical features of the anatomy of interest in the two-dimensional imaging using at least one machine learning model; determining characteristics of the plurality of anatomical features based on the detection of the plurality of anatomical features; and generating at least one measurement of the anatomy of interest based on at least some of the characteristics of the plurality of anatomical features.

### CROSS-REFERENCE TO RELATED APPLICATIONS

This application claims the benefit of U.S. Provisional Application No. 63/292,412, filed Dec. 21, 2021, the entire contents of which are hereby incorporated by reference herein.

### FIELD

This disclosure relates generally to orthopedics, and more particularly to image-based analysis of a joint.

### BACKGROUND

Orthopedics is a medical specialty that focuses on the diagnosis, correction, prevention, and treatment of patients with skeletal conditions, including for example conditions or disorders of the bones, joints, muscles, ligaments, tendons, nerves and skin, which make up the musculoskeletal system. Joint injuries or conditions such as those of the hip joint or other joints can occur from overuse or over-stretching or due to other factors, including genetic factors that may cause deviations from “normal” joint morphology.

Joints are susceptible to a number of different pathologies (e.g., conditions or disorders, which may cause deviation from the normal joint morphology). These pathologies can have both congenital and injury-related origins. In some cases, the pathology can be substantial at the outset. In other cases, the pathology may be minor at the outset but, if left untreated, may worsen over time. More particularly, in many cases an existing pathology may be exacerbated, for example, by the dynamic nature of the joint, the substantial weight loads imposed on the joint, or a combination thereof. The pathology may, either initially or thereafter, significantly interfere with patient comfort and lifestyle and may require surgical treatment.

The current trend in orthopedic surgery is to treat joint pathologies using minimally-invasive techniques such as joint arthroscopy in which an endoscope is inserted into the joint through a small incision. Procedures performed arthroscopically include debridement of bony pathologies in which portions of bone in a joint that deviate from a “normal” or target morphology are removed. During a debridement procedure, the surgeon uses an endoscopic camera to view the debridement area, but because the resulting endoscopic image has a limited field of view and is somewhat distorted, the surgeon cannot view the entire pathology all at once. As a result, it is generally quite difficult for the surgeon to determine exactly how much bone should be removed, and whether the shape of the remaining bone has the desired geometry.

### SUMMARY

According to an aspect, systems and methods can be used to generate measurements of anatomy of interest in two-dimensional imaging using machine learning models configured to detect anatomical features in the imaging. Characteristics of the anatomical features can be determined based on the detection of the features by the machine learning model and those characteristics can be used to generate measurements of the anatomy of interest, or the measurements may be generated by the machine learning model directly. The measurements may be displayed to a user for guidance in treatment and/or may be used to generate additional guidance for the user. Additionally or alternatively, a machine learning model may be trained to determine a morphological classification of the anatomy of interest.

According to an aspect, a method of generating a measurement of anatomy of interest from two-dimensional imaging includes receiving two-dimensional imaging associated with anatomy of interest, detecting a plurality of anatomical features of the anatomy of interest in the two-dimensional imaging using at least one machine learning model, determining characteristics of the plurality of anatomical features based on the detection of the plurality of anatomical features, and generating at least one measurement of the anatomy of interest based on at least some of the characteristics of the plurality of anatomical features.

Optionally, determining the characteristics of the plurality of anatomical features comprises determining an initial estimate of a characteristic of a first anatomical feature based on the detection of the plurality of anatomical features and determining a final estimate of the characteristic of the first anatomical feature based on the first estimate. The initial estimate of the characteristic of the first anatomical feature may include an estimate of at least one of a location and a size of the first anatomical feature, and determining the final estimate may include searching for a perimeter of the first anatomical feature based on the estimate of at least one of the location and the size of the first anatomical feature.

Optionally, the plurality of anatomical features comprises a head and neck of the femur and the characteristics comprise a location of mid-line of the neck.

Optionally, the at least one measurement comprises an Alpha Angle generated based on the location of the mid-line. The method may further include automatically generating a resection curve based on the Alpha Angle.

Optionally, the plurality of anatomical features detected comprises a plurality of features of a femur and the at least one measurement comprises an orientation of the femur relative to a predefined femur orientation. The method may further include determining an alignment of a three-dimensional model of the femur with the two-dimensional imaging based on the orientation of the femur.

Optionally, the method further includes comparing the orientation to a predefined orientation threshold and, in response to determining that the orientation is beyond the predefined orientation threshold, notifying the user.

Optionally, the at least one machine learning model generates a plurality of scored bounding boxes for the plurality of anatomical features and the characteristics of the plurality of anatomical features are determined based on bounding boxes that have scores that are above a predetermined threshold.

Optionally, the method further includes displaying a visual guidance associated with the anatomy of interest based on the at least one measurement. The visual guidance can provide, for example, guidance for bone treatment.

Optionally, the plurality of anatomical features detected comprises a plurality of features of a pelvis and the at least one measurement comprises an orientation of the pelvis relative to a predefined pelvis orientation.

Optionally, the method further includes comparing the orientation to a predefined orientation threshold and, in response to determining that the orientation is beyond the predefined orientation threshold, notifying the user.

Optionally, the at least one measurement of the anatomy of interest is generated using a regression machine learning model.

According to an aspect, a method of generating a measurement of anatomy of interest from two-dimensional imaging includes receiving two-dimensional imaging of a patient that comprises the anatomy of interest; and generating at least one measurement of the anatomy of interest using a machine learning model trained based on a plurality of two-dimensional images that have been tagged with corresponding measurements of the anatomy of interest.

Optionally, the plurality of two-dimensional images comprises a plurality of pseudo two-dimensional images generated from at least one three-dimensional imaging data set.

Optionally, the anatomy of interest comprises a femur or a pelvis and the measurement comprises an orientation of the femur or pelvis.

Optionally, the anatomy of interest is a hip joint and the at least one measurement comprises Alpha Angle, head-neck offset, Center Edge Angle, Tönnis angle, acetabular version, femoral version, acetabular coverage, or femoral neck shaft angle.

Optionally, the method further includes displaying a visual guidance associated with the anatomy of interest based on the at least one measurement. The visual guidance can provide, for example, guidance for bone treatment.

Optionally, the at least one measurement comprises at least one pelvic orientation, and generating the at least one measurement comprises detecting an obturator foramen and determining the at least one pelvic orientation based on the obturator foramen.

Optionally, determining the at least one measurement comprises analyzing the obturator foramen using a regression machine learning model.

According to an aspect, a method for determining a morphological classification of anatomy of interest includes receiving two-dimensional imaging of a patient that comprises the anatomy of interest; and determining the morphological classification of the anatomy of interest using at least one machine learning classifier trained to identify different morphological classifications.

Optionally, the anatomy of interest is a hip and the morphological classification comprises a posterior wall sign, a crossover sign, an ischial spine sign, an acetabular cup depth, a Shenton's line, and a teardrop sign.

According to an aspect, a system includes one or more processors, memory, and one or more programs stored in the memory for execution by the one or more processors for causing the system to perform any of the preceding methods.

According to an aspect, a non-transitory computer readable medium stores instructions for execution by one or more processors of a system to cause the system to perform any of the above methods.

It will be appreciated that any of the variations, aspects, features and options described in view of the systems apply equally to the methods and vice versa. It will also be clear that any one or more of the above variations, aspects, features and options can be combined.

### BRIEF DESCRIPTION OF THE FIGURES

The invention will now be described, by way of example only, with reference to the accompanying drawings, in which:

FIGS. 1A-1D are schematic views showing various aspects of hip motion;

FIG. 2 is a schematic view showing bone structures in the region of the hip joint;

FIG. 3 is a schematic anterior view of the femur;

FIG. 4 is a schematic posterior view of the top end of the femur;

FIG. 5 is a schematic view of the pelvis;

FIGS. 6-12 are schematic views showing bone and soft tissue structures in the region of the hip joint;

FIGS. 13A and 13B are schematic views showing cam-type femoroacetabular impingement;

FIGS. 14A and 14B are schematic views showing pincer-type femoroacetabular impingement;

FIG. 15 is a schematic view showing a labral tear;

FIG. 16 is a schematic view showing an Alpha Angle determination on the hip of a patient;

FIG. 17 is a schematic view showing a Center Edge Angle determination on a hip of a patient;

FIG. 18 is a schematic view of an exemplary surgical suite;

FIG. 19 illustrates an exemplary method for generating one or more measurements of anatomy of interest from two-dimensional imaging;

FIG. 20 illustrates an example of scored bounding boxes generated via a machine learning model trained to detect the head and neck of a femur;

FIG. 21 is a block diagram of a method for determining an Alpha Angle based on anatomical features detected by a machine learning model;

FIG. 22 is an exemplary X-ray image with estimates for the centers of the femoral head and neck;

FIG. 23 illustrates an example of the results of an edge detection algorithm for a femoral head;

FIG. 24 illustrates an example of a circle from a Hough transform encircling the edges of the femoral head detected via edge detection;

FIG. 25 illustrates an example of the determination of where the femoral head stops being round and a cam pathology starts;

FIG. 26 is a schematic view showing one way of measuring the Alpha Angle;

FIG. 27 is a schematic view showing an exemplary resection curve for treating cam-type femoroacetabular impingement;

FIG. 28 is a schematic view showing aspects of the generation of a resection curve for treating cam-type femoroacetabular impingement;

FIG. 29 is an exemplary graphical user interface for providing a user with a resection curve based on head-neck offset measurements;

FIG. 30 is a schematic view showing an example of a Center Edge Angle calculation;

FIG. 31 illustrates an example of features of a femur detected by a machine learning model that can be used to determine an orientation of the femur;

FIG. 32 is a block diagram of an exemplary method that uses one or more machine learning models to generating at least one measurement of anatomy of interest from two-dimensional imaging;

FIG. 33 is a diagram of an exemplary method for determining a morphological classification of anatomy of interest;

FIG. 34 and FIG. 35 illustrate different morphological classifications for a hip joint that may be identified according to the method of FIG. 33; and

FIG. 36 is a block diagram of an exemplary computing system.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

According to an aspect, systems and methods include using machine learning for automatically determining a variety of clinically relevant measurements and classifications of anatomy of interest from two-dimensional imaging. The systems and methods enable automated measurements and/or characterizations that may be difficult to perform by hand, particularly intraoperatively. Additionally, the automatic generation of measurements and/or characterizations can provide improved accuracy and performance compared to manual determinations, as well as minimizing the need for user input or actions.

Some conventional computer-aided image analysis systems are available that offer annotation-like tools to generate measurements in imaging, but these systems generally require heavy user involvement. For example, a user may be asked to provide an input with respect to a displayed image indicating the locations of various portions of the anatomy from which a measurement can be generated. This user involvement can be quite burdensome, particularly when required intraoperatively, and user input is prone to human error. Additionally, some measurements and classifications are determined entirely by hand and require proper anatomical positioning and imaging views, which a user may not be able to verify from the imaging. Thus, conventional solutions have not worked well because they involve too much user input or may be may be too difficult for a human to determine.

The systems and methods described herein automate the generation of measurements and classification, greatly reducing or eliminating user involvement and enabling measurements and classifications that may not have been previously possible by hand. These advantages can make the generation of measurements and/or classification more readily available to users, which can improve patient outcomes, such as when used for treatment planning and/or treatment assessment, preoperatively, intraoperatively, and/or postoperatively.

Although the following examples often refer to hip joints, hip joint pathologies, and hip joint characteristics and measurements, it is to be understood that the systems, methods, techniques, visualizations, etc., described herein according to various embodiments, can be used for analyzing and visualizing other joints, including knees, shoulders, elbows, the spine, the ankle, etc.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some embodiments also relates to a device for performing the operations herein. This device may be specially constructed for the required purposes, or it may comprise a general-purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

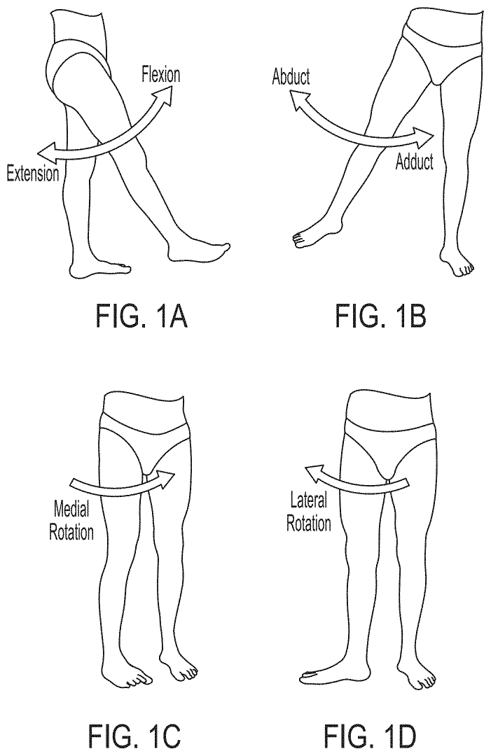

A better understanding of various joint pathologies, and the advantages provided according to the systems and methods described herein, can be gained from the following description of the anatomy of the joint. The hip joint is formed at the junction of the femur and the hip. The hip joint is a ball-and-socket joint, and is capable of a wide range of different motions, e.g., flexion and extension, abduction and adduction, internal and external rotation, etc., as illustrated in FIGS. 1A-1D. With the possible exception of the shoulder joint, the hip joint is perhaps the most mobile joint in the body. The hip joint carries substantial weight loads during most of the day, in both static (e.g., standing and sitting) and dynamic (e.g., walking and running) conditions.

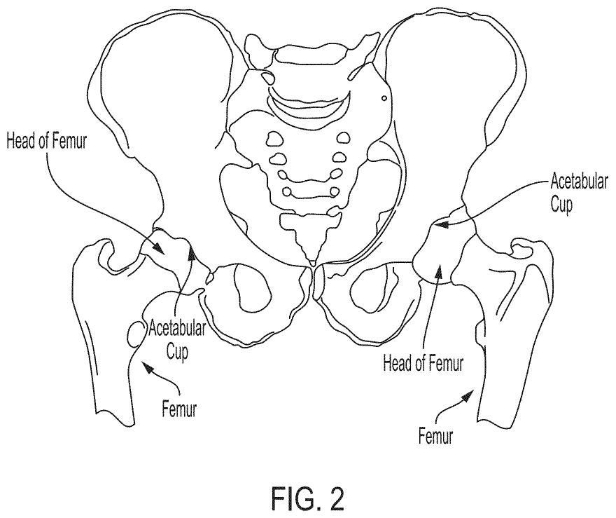

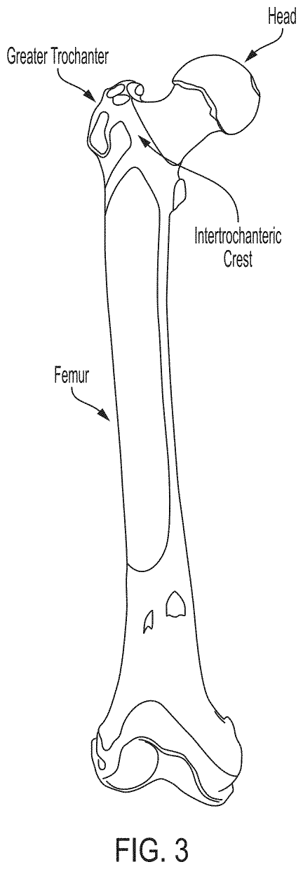

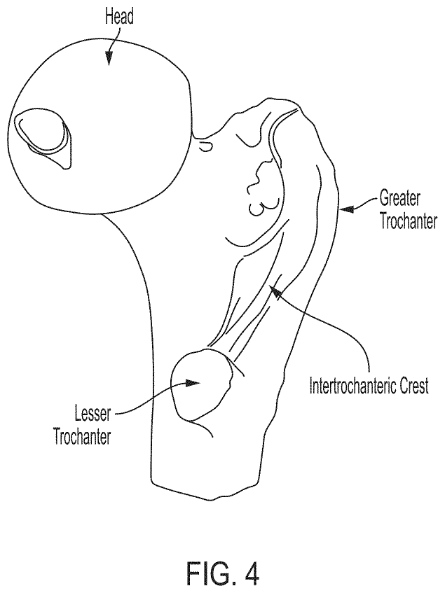

More particularly, and with reference to FIG. 2, the ball of the femur is received in the acetabular cup of the hip, with a plurality of ligaments and other soft tissue serving to hold the bones in articulating condition. As is illustrated in FIG. 3, the femur is generally characterized by an elongated body terminating, at its top end, in an angled neck which supports a hemispherical head (also sometimes referred to as the ball). As is illustrated in FIGS. 3 and 4, a large projection known as the greater trochanter protrudes laterally and posteriorly from the elongated body adjacent to the neck. A second, somewhat smaller projection known as the lesser trochanter protrudes medially and posteriorly from the elongated body adjacent to the neck. An intertrochanteric crest extends along the periphery of the femur, between the greater trochanter and the lesser trochanter.

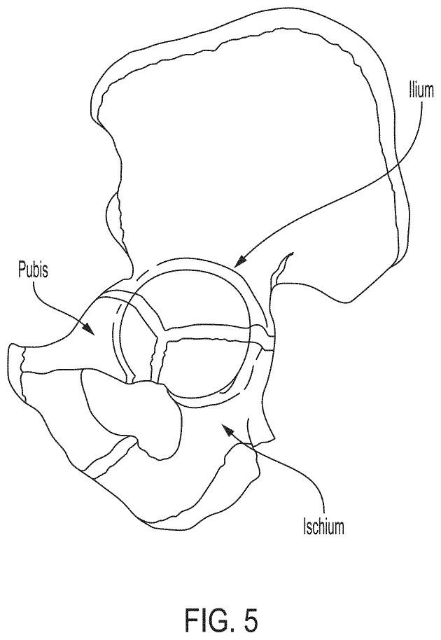

Referring to FIG. 5, the pelvis is made up of three constituent bones: the ilium, the ischium and the pubis. These three bones cooperate with one another (they typically ossify into a single “hip bone” structure by the age of 25) so as to form the acetabular cup. The acetabular cup receives the head of the femur.

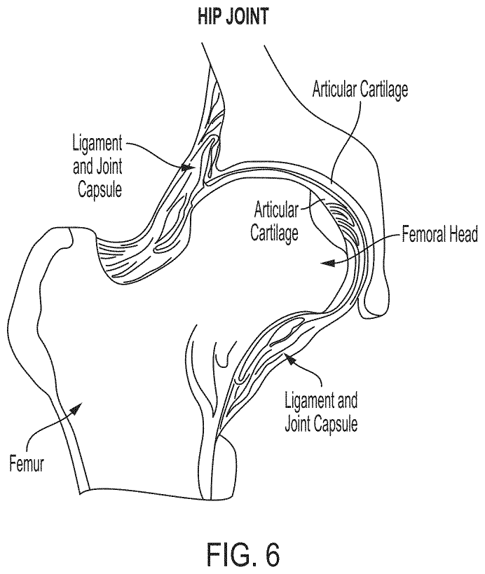

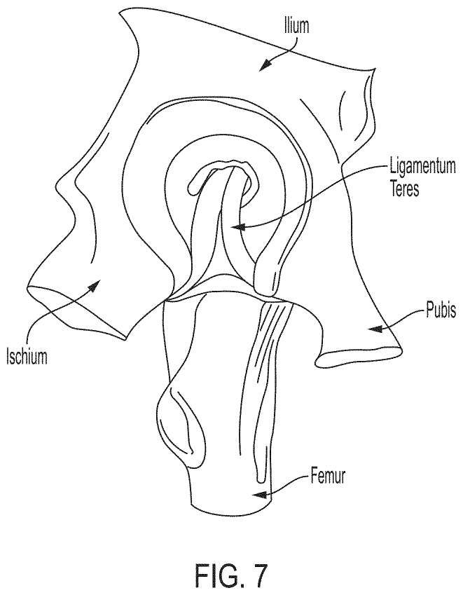

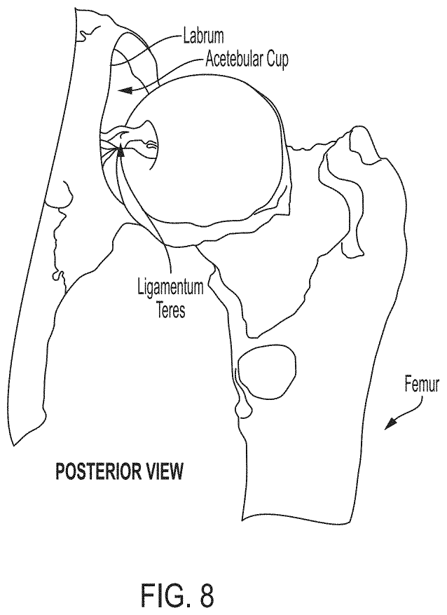

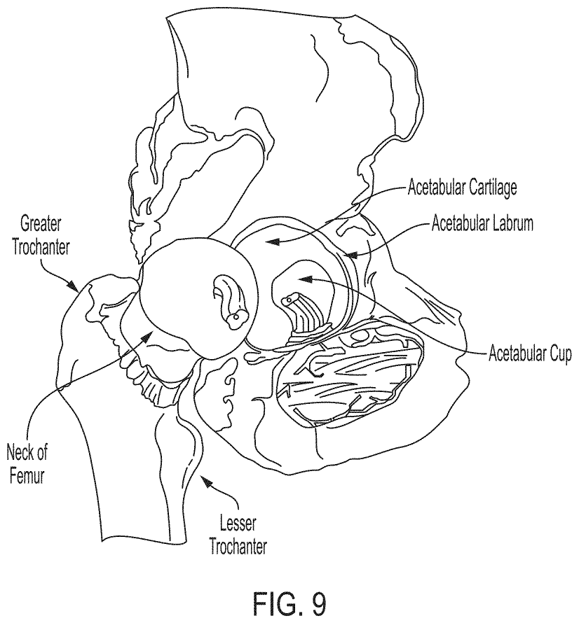

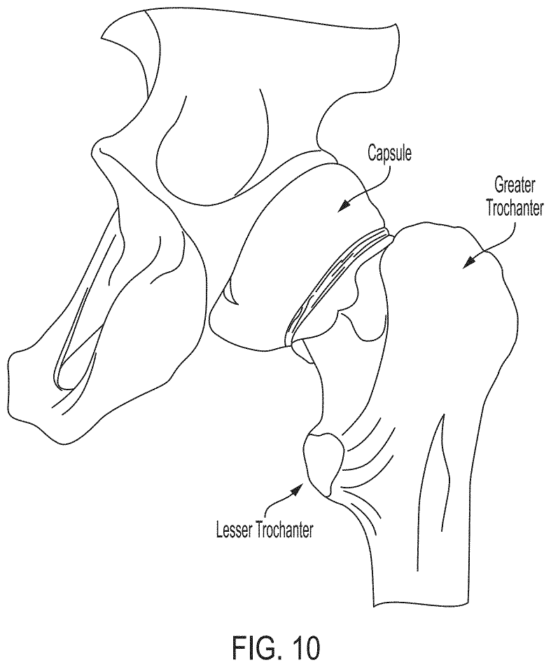

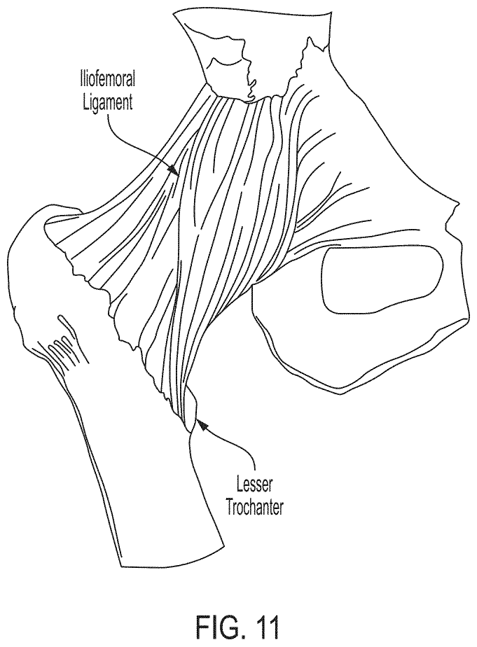

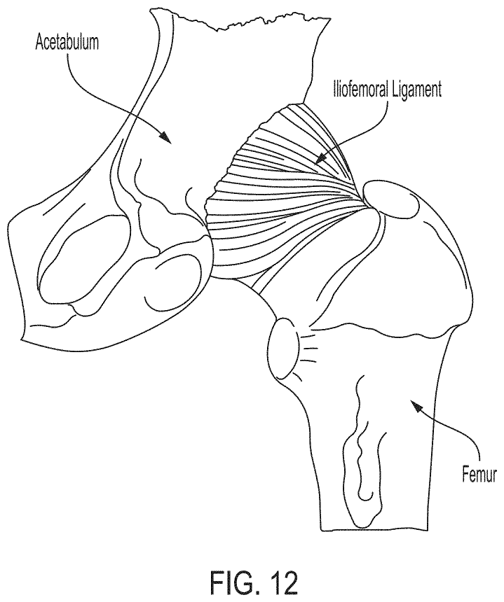

Both the head of the femur and the acetabular cup are covered with a layer of articular cartilage which protects the underlying bone and facilitates motion (see FIG. 6). Various ligaments and soft tissue serve to hold the ball of the femur in place within the acetabular cup. More particularly, and with reference to FIGS. 7 and 8, the ligamentum teres extends between the ball of the femur and the base of the acetabular cup. Referring to FIG. 9, a labrum is disposed about the perimeter of the acetabular cup. The labrum serves to increase the depth of the acetabular cup and effectively establishes a suction seal between the ball of the femur and the rim of the acetabular cup, thereby helping to hold the head of the femur in the acetabular cup. In addition, and with reference to FIG. 10, a fibrous capsule extends between the neck of the femur and the rim of the acetabular cup, effectively sealing off the ball-and-socket members of the hip joint from the remainder of the body. The foregoing structures are encompassed and reinforced by a set of three main ligaments (i.e., the iliofemoral ligament, the ischiofemoral ligament and the pubofemoral ligament) which extend between the femur and the hip (see FIGS. 11 and 12).

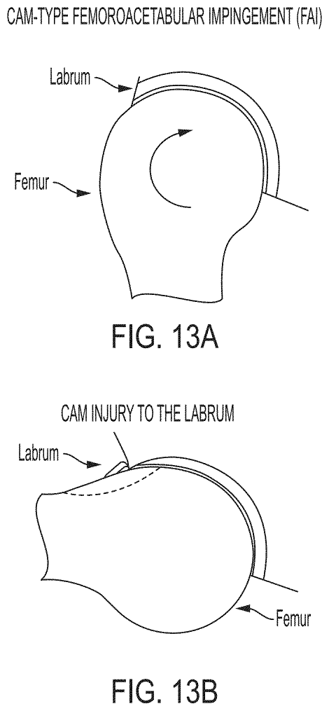

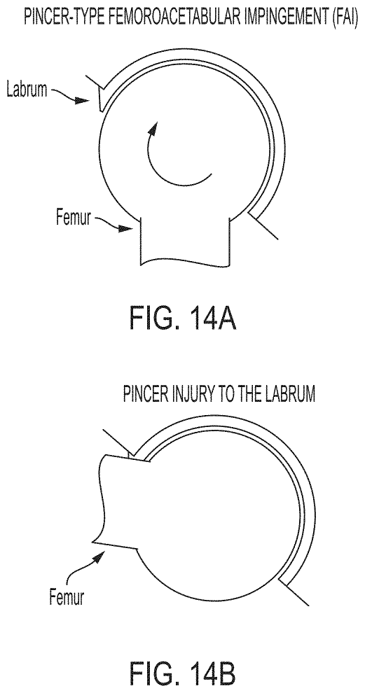

The hip joint is susceptible to a number of different pathologies. These pathologies can have, for example, both congenital and injury-related origins. For example, a congenital pathology of the hip joint involves impingement between the neck of the femur and the rim of the acetabular cup. In some cases, and with reference to FIGS. 13A and 13B, this impingement can occur due to irregularities in the geometry of the femur. This type of impingement is sometimes referred to as a cam-type femoroacetabular impingement (i.e., a cam-type FAI). In other cases, and with reference to FIGS. 14A and 14B, the impingement can occur due to irregularities in the geometry of the acetabular cup. This latter type of impingement is sometimes referred to as a pincer-type femoroacetabular impingement (i.e., a pincer-type FAI). Impingement can result in a reduced range of motion, substantial pain and, in some cases, significant deterioration of the hip joint.

Another example of congenital pathology of the hip joint involves defects in the articular surface of the ball and/or the articular surface of the acetabular cup. Defects of this type sometimes start out fairly small but often increase in size over time, generally due to the dynamic nature of the hip joint and also due to the weight-bearing nature of the hip joint. Articular defects can result in substantial pain, induce or exacerbate arthritic conditions and, in some cases, cause significant deterioration of the hip joint.

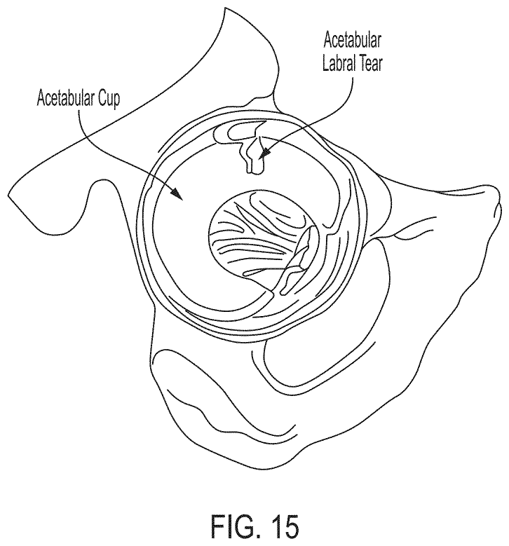

An example of injury-related pathology of the hip joint involves trauma to the labrum. In many cases, an accident or a sports-related injury can result in the labrum being torn, typically with a tear running through the body of the labrum (e.g., see FIG. 15). These types of injuries can be painful for the patient and, if left untreated, can lead to substantial deterioration of the hip joint.

The current trend in orthopedic surgery is to treat joint pathologies using minimally-invasive techniques. For example, it is common to re-attach ligaments in the shoulder joint using minimally-invasive, “keyhole” techniques which do not require “laying open” the capsule of the shoulder joint. Furthermore, it is common to repair, for example, torn meniscal cartilage in the knee joint, and/or to replace ruptured ACL ligaments in the knee joint, using minimally-invasive techniques. While such minimally-invasive approaches can require additional training on the part of the surgeon, such procedures generally offer substantial advantages for the patient and have now become the standard of care for many shoulder joint and knee joint pathologies.

In addition to the foregoing, due to the widespread availability of minimally-invasive approaches for treating pathologies of the shoulder joint and knee joint, the current trend is to provide such treatment much earlier in the lifecycle of the pathology, so as to address patient pain as soon as possible and so as to minimize any exacerbation of the pathology itself. This is in marked contrast to traditional surgical practices, which have generally dictated postponing surgical procedures as long as possible so as to spare the patient from the substantial trauma generally associated with invasive surgery.

Minimally-invasive treatments for pathologies of the hip joint have lagged behind minimally-invasive treatments for pathologies of the shoulder joint and knee joint. This may be, for example, due to (i) the geometry of the hip joint itself, and (ii) the nature of the pathologies which must typically be addressed in the hip joint.

The hip joint is generally considered to be a “tight” joint, in the sense that there is relatively little room to maneuver within the confines of the joint itself. This is in contrast to the knee joint, which is generally considered to be relatively spacious when compared to the hip joint. As a result, it is relatively more challenging for surgeons to perform minimally-invasive procedures on the hip joint.

Furthermore, the natural pathways for entering the interior of the hip joint (i.e., the pathways which naturally exist between adjacent bones) are generally much more constraining for the hip joint than for the shoulder joint or the knee joint. This limited access further complicates effectively performing minimally-invasive procedures on the hip joint.

In addition to the foregoing, the nature and location of the pathologies (e.g., conditions or disorders, which may cause deviation from the baseline anatomy of the joint) of the hip joint also complicate performing minimally-invasive procedures. For example, in the case of a typical tear of the labrum in the hip joint, instruments must generally be introduced into the joint space using a line of approach which is set, in some locations, at an angle of 25 degrees or more to the line of repair. This makes drilling into bone, for example, much more complex than where the line of approach is effectively aligned with the line of repair, such as is frequently the case in the shoulder joint. Furthermore, the working space within the hip joint is typically extremely limited, further complicating repairs where the line of approach is not aligned with the line of repair.

As a result of the foregoing, minimally-invasive hip joint procedures continue to be relatively difficult, and patients must frequently manage their hip joint pathologies for as long as possible, until a partial or total hip replacement can no longer be avoided, whereupon the procedure is generally done as a highly-invasive, open procedure, with all of the disadvantages associated with highly-invasive, open procedures.

As noted above, hip arthroscopy is becoming increasingly more common in the diagnosis and treatment of various hip pathologies. However, due to the anatomy of the hip joint and the pathologies associated with the same, hip arthroscopy appears to be currently practical for only selected pathologies and, even then, hip arthroscopy has generally met with limited success.

One procedure which is sometimes attempted arthroscopically relates to femoral debridement for treatment of cam-type femoroacetabular impingement (i.e., cam-type FAI). More particularly, with cam-type femoroacetabular impingement, irregularities in the geometry of the femur can lead to impingement between the femur and the rim of the acetabular cup. Treatment for cam-type femoroacetabular impingement typically involves debriding the femoral neck and/or head, using instruments such as burrs and osteotomes, to remove the bony deformities causing the impingement. It is important to debride the femur carefully, since only bone which does not conform to the desired geometry should be removed, in order to ensure positive results as well as to minimize the possibility of bone fracture after treatment. For this reason, when debridement is performed as an open surgical procedure, surgeons generally use debridement templates having a pre-shaped curvature to guide them in removing the appropriate amount of bone from the femur.

However, when the debridement procedure is attempted arthroscopically, conventional debridement templates with their pre-shaped curvature cannot be passed through the narrow keyhole incisions, and hence debridement templates are generally not available to guide the surgeon in reshaping the bone surface. As a result, the debridement must generally be effected “freehand.” In addition to the foregoing, the view of the cam pathology is also generally limited. Primarily, the surgeon uses a scope and camera to view the resection area, but the scope image has a limited field of view and is somewhat distorted. Also, because the scope is placed close to the bone surface, the surgeon cannot view the entire pathology “all at once.” Secondarily, the surgeon also utilizes a fluoroscope to take X-ray images of the anatomy. These X-ray images supplement the arthroscopic view from the scope, but it is still limited to a two-dimensional representation of the three-dimensional cam pathology.

As a result of the foregoing, it is generally quite difficult for the surgeon to determine exactly how much bone should be removed, and whether the shape of the remaining bone has the desired geometry. In practice, surgeons tend to err on the side of caution and remove less bone. Significantly, under-resection of the cam pathology is the leading cause of revision hip arthroscopy.

An example of another procedure which is sometimes attempted arthroscopically relates to treatment of pincer-type femoroacetabular impingement (i.e., pincer-type FAI). More particularly, with pincer-type femoroacetabular impingement, irregularities in the geometry of the acetabulum can lead to impingement between the femur and the rim of the acetabular cup. Treatment for pincer-type femoroacetabular impingement typically involves debriding the rim of the acetabular cup using instruments such as burrs and osteotomes to remove the bony deformities causing the impingement. In some cases, the labrum is released from the acetabular bone so as to expose the underlying rim of the acetabular cup prior to debriding the rim of the acetabular cup, and then the labrum is reattached to the debrided rim of the acetabular cup. It is important to debride the rim of the acetabular cup carefully, since only bone which does not conform to the desired geometry should be removed, in order to alleviate impingement while minimizing the possibility of removing too much bone from the rim of the acetabular cup, which could cause joint instability.

However, when the debridement procedure is attempted arthroscopically, the debridement must generally be effected freehand. In this setting, it is generally quite difficult for the surgeon to determine exactly how much bone should be removed, and whether the remaining bone has the desired geometry. In practice, surgeons tend to err on the side of caution and remove less bone. Significantly, under-resection of the pincer pathology may necessitate revision hip arthroscopy.

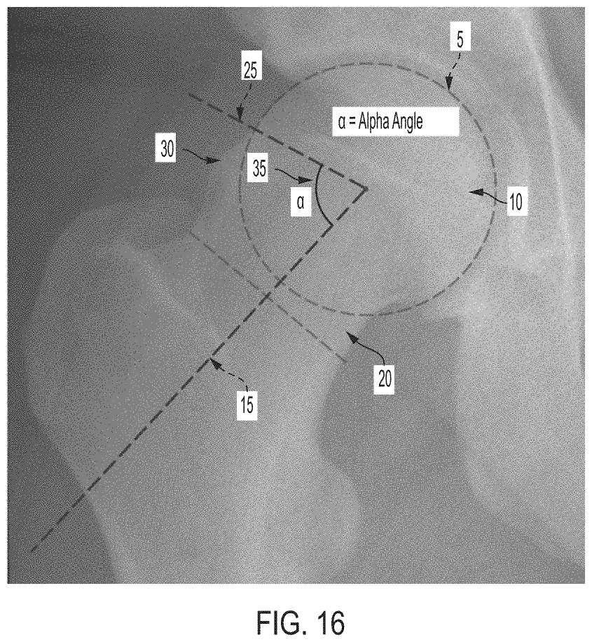

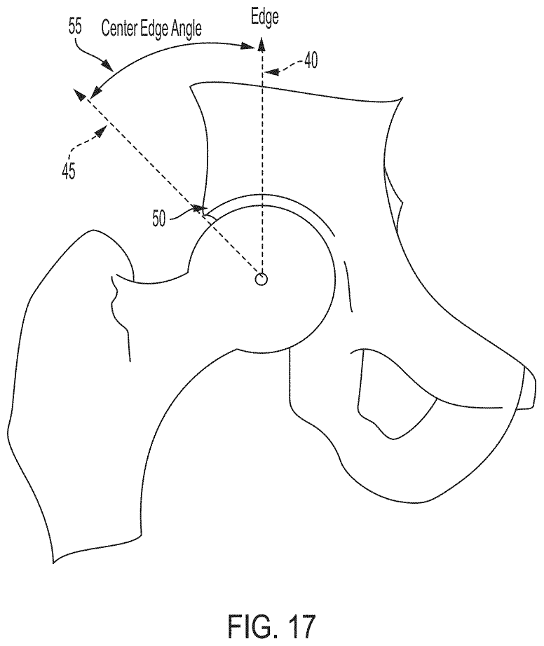

Two common anatomical measurements used in diagnosing femoroacetabular impingement (FAI) are the Alpha Angle (FIG. 16) for cam-type impingement and the Center Edge Angle (FIG. 17) for pincer-type impingement. These measurements are typically measured from pre-operative images (e.g., pre-operative X-ray images). These measurements are used to determine the degree to which the patient's hip anatomy deviates from normal (e.g., baseline), healthy hip anatomy.

For example, a healthy hip typically has an Alpha Angle of anywhere from less than approximately 42 degrees to approximately 50 degrees; thus, a patient with an Alpha Angle of greater than approximately 42 degrees to approximately 50 degrees may be a candidate for FAI surgery. These are merely exemplary Alpha Angle ranges and do not limit the systems and methods herein to any particular range of Alpha Angles. During an initial examination of a patient, the surgeon will typically take an X-ray of the patient's hip. If the patient has an initial diagnosis of FAI, the patient may also obtain an MRI or CT scan of their hip for further evaluation of the bony pathology causing the FAI.

Most of today's imaging techniques (e.g., X-ray, CT, MM) are digital, and hence the images can be imported into, and manipulated by, computer software. Using the imported digital images, the surgeon is able to measure the Alpha Angle (and/or the Center Edge Angle). For example, the surgeon imports the digital image into one of the many available software programs that use the DICOM (Digital Imaging and Communications in Medicine) standard for medical imaging. In order to make the Alpha Angle (or the Center Edge Angle) measurements with the digital image, the surgeon must first manually create and overlay geometric shapes onto the digital medical image.

For example, and with reference to FIG. 16, to measure the Alpha Angle conventionally, a surgeon may manually create a circle 5 and place it over the femoral head 10, and then manually size the circle such that the edge of the circle matches the edge of the femoral head. The surgeon then manually creates a line 15 and places it along the mid-line of the femoral neck 20. The surgeon then manually draws a second line 25 which originates at the center of the femoral head and passes through the location which signifies the start of the cam pathology 30 (i.e., the location where the bone first extends outside the circle set around the femoral head). The surgeon then manually selects the two lines and instructs the software to calculate the angle between the two lines; the result is the Alpha Angle 35.

Correspondingly, and with reference to FIG. 17, to measure the Center Edge Angle, the surgeon manually creates a vertical line 40 which originates at the center of the femoral head and is perpendicular to the transverse pelvic axis. The surgeon then manually draws a second line 45 which originates at the center of the femoral head and passes through the location which signifies the start of the pincer pathology 50 (i.e., the rim of the acetabular cup). The surgeon then manually selects the two lines and instructs the software to calculate the angle between the two lines; the result is the Center Edge Angle 55.

These Alpha Angle measurements (or Center Edge Angle measurements) are typically performed around the time that the patient is initially examined, which typically occurs weeks or months prior to surgery. At the time of surgery, the surgeon may bring a copy (e.g., a printout) of the Alpha Angle measurements (or the Center Edge Angle measurements) to the operating room so that the printout is available as a reference during surgery. The surgeon may also have access to these measurements with a computer located in or near the operating room, which is connected to the hospital's PACS system (Picture Archiving and Communication System). Either way, the surgeon can have the pre-operative measurements available as a reference during surgery.

However, while the surgeon is debriding bone on the cam (or pincer), the-pre-operative measurements may be insufficient for adequately guiding the surgeon regarding where and how much bone should be removed due to the difficulty in comparing what the surgeon sees in the endoscopic images with the pre-operative measurements. Accordingly, as discussed further below with respect to various embodiments, systems and methods can guide a surgeon during a surgical procedure on a joint by displaying an overlay of a three-dimensional representation of planned bone removal on a two-dimensional image of the joint captured during the surgical procedure. The three-dimensional representation of planned bone removal can indicate where bone should be removed from the joint in three-dimensional space, so that the surgeon can better understand how the planned bone removal relates to what the surgeon is seeing via the endoscopic imaging.

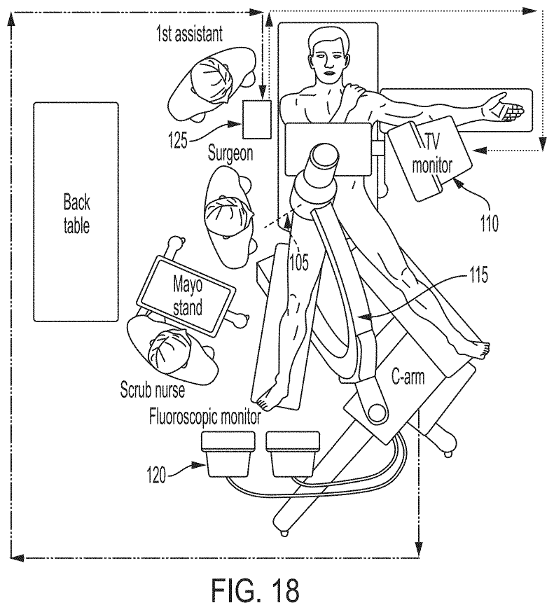

FIG. 18 illustrates a surgical suite incorporating a system for guiding a surgeon in removing bone from a portion of a joint during a surgical procedure, according to some embodiments. In a typical arthroscopic surgical suite, the surgeon uses an arthroscope 105 and a display 110 to directly view an internal surgical site. In addition, the surgeon may also use a C-arm X-ray machine 115 and a fluoroscopic display 120 to image the internal surgical site. In accordance with various embodiments, the surgical suite can include a visual guidance system 125 that can generate an overlay image in which a representation of bone removal extracted from a three-dimensional model of the bone is overlaid on a two-dimensional image of the bone captured intra-operatively, such as by a C-arm X-ray machine 115, according to the principles described herein, for guiding the surgeon during the surgical procedure.

According to some embodiments, visual guidance system 125 comprises one or more processors, memory, and one or more programs stored in the memory for causing the visual guidance system to provide the functionality disclosed herein. According to some embodiments, visual guidance system 125 comprises a tablet device with an integrated computer processor and user input/output functionality, e.g., a touchscreen. The visual guidance system 125 may be at least partially located in the sterile field, for example, the visual guidance system 125 may comprise a touchscreen tablet mounted to the surgical table or to a boom-type tablet support. The visual guidance system 125 may be covered by a sterile drape to maintain the surgeon's sterility as he or she operates the touchscreen tablet. Visual guidance system 125 may comprise other general purpose computers with appropriate programming and input/output functionality, e.g., a desktop or laptop computer with a keyboard, mouse, touchscreen display, heads-up display, gesture recognition device, voice activation feature, pupil reading device, etc.

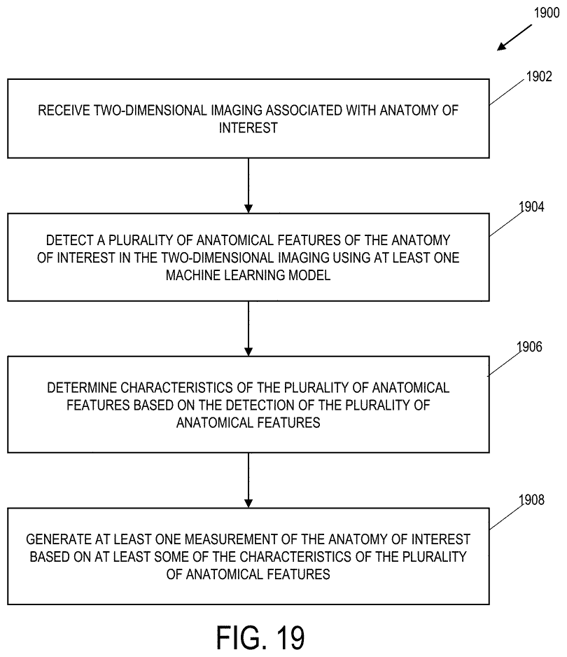

FIG. 19 illustrates an exemplary method 1900 for generating one or more measurements of anatomy of interest from two-dimensional imaging. Method 1900 can be performed before, during, and/or after a medical procedure, such as a surgical procedure or a non-surgical procedure, by a visual guidance system, such as visual guidance system 125 of FIG. 18. The measurements can be displayed to a surgeon or other medical practitioner and/or can be used to generate a visual guidance for the surgeon, such as for guiding a treatment.

At step 1902, two-dimensional imaging associated with anatomy of interest is received at a computing system. The two-dimensional imaging can include one or more single snapshot images and/or video frames. The two-dimensional imaging generally includes anatomy of interest of a patient. For example, the two-dimensional imaging may include a portion of bone that is being or will be surgically treated as well as surrounding portions of the bone that enable the surgeon to generally compare what is shown in the image to what the surgeon is seeing endoscopically. For example, in embodiments involving debridement to address a CAM pathology, the two-dimensional image generally includes the head and neck of the femur. The image may further include the greater and/or lesser trochanter of the femur, which may ensure that a sufficient portion of the femur is visible for generating the one or more measurements. The two-dimensional image can be received from an intra-operative imaging system, such as an X-ray imager (e.g., C-arm X-ray machine 115 of FIG. 18) that is communicatively connected with the computing system performing method 1900. Optionally, one or more pre-processing operations are applied to the two-dimensional imaging, such as one or more scaling operations, cropping operations, down-sampling, up-sampling, etc. Optionally, a dewarping operation is applied to an X-ray image to correct for warping caused by the imaging system. Dewarping of an X-ray image can be performed based on the determined relationship between a known pattern of reference markers attached to the detector of the imaging system and the reference markers visible in the X-ray image. For example, the reference markers in an X-ray image may be detected, a non-rigid transformation that maps the known positions of the reference markers to the markers visible in the image may be calculated, and the transformation may be applied to the image, resulting in a dewarped image. The reference markers may then be removed from the image.

At step 1904, a plurality of anatomical features of the anatomy of interest are detected in the two-dimensional imaging using at least one machine learning model. The machine learning model can be an object detection model trained to detect one or more features of the anatomy in two-dimensional imaging. For example, an object detection machine learning model can be trained to detect the femoral head and/or femoral neck or any other portions of a femur, such as the greater trochanter, lesser trochanter, and/or femoral shaft. With respect to a pelvis, an object detection machine learning model can be trained to detect an anterior acetabular rim, a posterior acetabular rim, an illiopectineal line, an illioischial line, an acetabular roof, an acetabulum, an obturator foramen, and/or a pubic symphysis. An object detection machine learning model could utilize a convolutional neural network (CNN), such as R-CNN or YOLO architecture, or any other suitable object detection model.

In some examples, the trained machine learning models may be re-trained using log images, which are images captured and automatically or manually logged/recorded during surgical procedures and subsequently made available for re-training. The log images may include images with one or more features making the image particularly difficult for the machine learning model to process. For instance, the log images may include one or more tools in the image that may partially block the anatomy of interest, images of slipped capital femoral epiphysis (SCFE), or oval-shaped (or otherwise uncommonly shaped) femoral heads, or any combination of these features.

The machine learning model can generate bounding boxes surrounding portions of the imaging along with a numerical score for each bounding box that corresponds to a confidence that the respective portion of the imaging includes the feature that the machine learning model is trained to detect. Machine learning models trained to detect multiple different features may also provide a feature classification for each bounding box. Thus, for example, a machine learning model analyzing two-dimensional imaging of a femur may output bounding boxes that including classifications for one or more of the head of the femur, the neck of the femur, the shaft of the femur, the greater trochanter, the lesser trochanter, etc., with each bounding box having a confidence score that the corresponding portion of the two-dimensional imaging includes the respective feature of the femur. Post processing of the machine learning model results may determine the highest scoring bounding box for each classification. The scores may be compared to one or more threshold values, and if the scores meet the threshold values, then the feature may be considered detected. If a given feature does not have a score that meets the threshold, then a warning or other notification may be provided to a user that the feature was not detecting in the imaging.

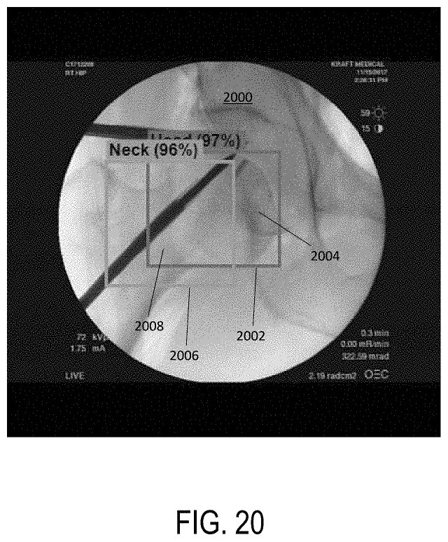

FIG. 20 illustrates an example of scored bounding boxes generated via a machine learning model trained to detect the head and neck of a femur, according to aspects of step 1904. The machine learning model provides a bounding box 2002 bounding the femoral head 2004 captured in an X-ray image 2000 and a bounding box 2006 bounding the femoral neck 2008 in the image 2000. The femoral head bounding box 2002 has a score of 97% and the femoral neck bounding box 2006 has a score of 96%. If these scores meet a predetermined threshold, then the bounding boxes may be selected as detections of the respective features.

At step 1906, one or more characteristics of the anatomical features detected in step 1904 are determined. The one or more characteristics can be determined directly from the bounding boxes from step 1904 and/or can be derived from information determined from the bounding boxes. For example, the center of each bounding box may be used as the center of the respective feature or may be used as a starting point for a further search for a center of the feature or another characteristic of the feature. Additionally or alternatively, the size of a bounding box can be used as a characteristic or for determining a characteristic. For example, a machine learning model may be trained to generate a bounding box that aligns with the outer bounds of a feature, and a dimension of the bounding box can be used as a dimension of the feature. Taking FIG. 20 as an example, bounding box 2002 may at least partially align with the outer perimeter of the femoral head 2004. This may be achieved by training a machine learning model to generate a bounding box that has sides that are near (e.g., tangent to) the outer perimeter of the femoral head. The distance from the center of the bounding box to one of its sides can then be used as the radius of the femoral head or can be used as a starting point for determining the radius of the femoral head or any other feature of the femoral head for which the radius may be useful.

Method 1900 may be used for imaging of a femur and examples of characteristics of features of a femur determined at step 1906 include one or more of a location of a femoral head, a location of a femoral neck, a radius and/or diameter of a femoral head, a location of a femoral shaft, a location of a greater trochanter, and/or a location of a lesser trochanter. Method 1900 may be used for imaging of a pelvis, and one or more characteristics of the features of the pelvis determined at step 1906 can include a location of the acetabulum, a location of the superior and inferior acetabular edges, a location of the obturator foramen, and/or a location of the pubic symphysis. Method 1900 may be applied to imaging of a knee joint, and the one or more characteristics can be a location of the tibial plateau, a location of the tibial shaft, and/or a location of the intercondylar eminence. Method 1900 can be applied to imaging of one or more vertebra, and characteristics of features of the vertebra can include a location of a pedicle, a location of a facet, a location of a superior endplate, and/or a location of an inferior endplate.

At step 1908, at least one measurement of the anatomy of interest is determined based on at least some of the characteristics of the plurality of anatomical features. For example, where the anatomy of interest is the hip, the at least one measurement could be or include, for example, an Alpha Angle, Center Edge Angle, a head-neck offset, Tönnis angle (also referred to as acetabular inclination and acetabular index), acetabular version, femoral torsion, femoral version, acetabular coverage, orientation of a femur and/or pelvis, and femoral neck shaft angle, just to name a few. Examples of generating some of these measurements are described below.

### Generating Alpha Angle According to Method 1900

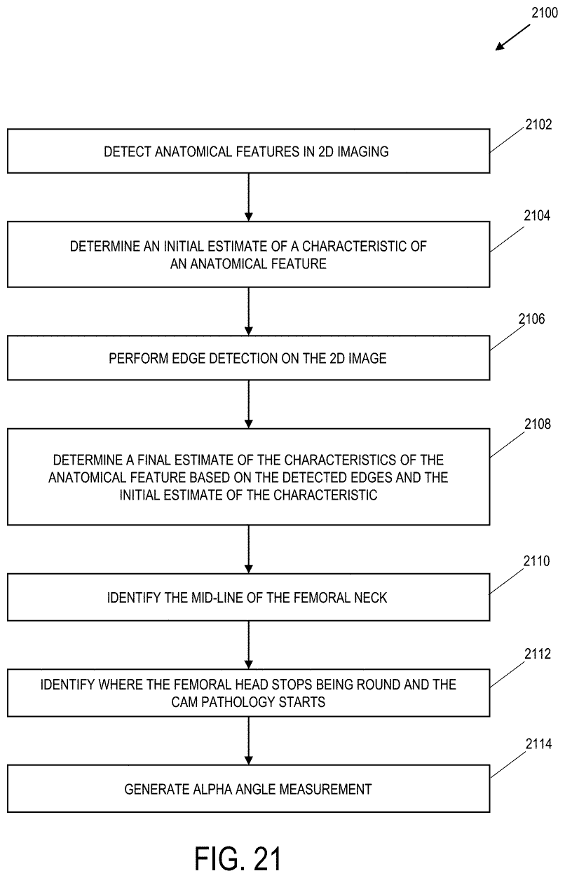

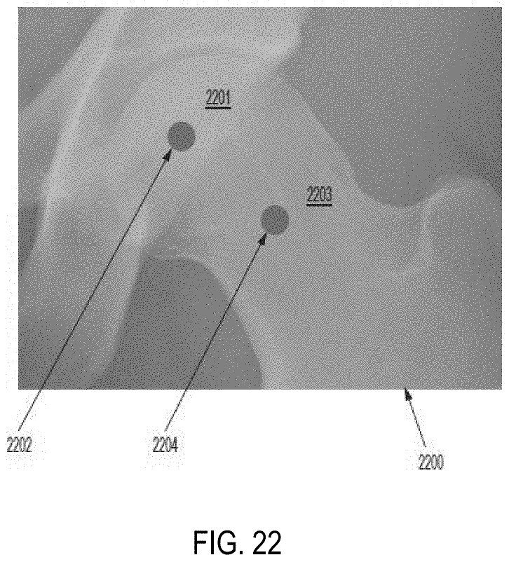

FIG. 21 is a block diagram of a method 2100 for determining an Alpha Angle based on anatomical features detected by a machine learning model, according to an example of method 1900. Method 2100 is performed by a computing system, such as visual guidance system 125. At step 2102, a machine learning model is used to detect one or more anatomical features in two-dimensional imaging, such as X-ray image 2200 of FIG. 22. With respect to FIG. 22, the detected features may include, for example, the femoral head 2201 and femoral neck 2203.

At step 2104, an initial estimate of one or more characteristics of the anatomical features is determined. For example, the centers of bounding boxes for a femoral head and femoral neck may be used as a femoral head center estimate 2202 and a femoral neck center estimate 2204.

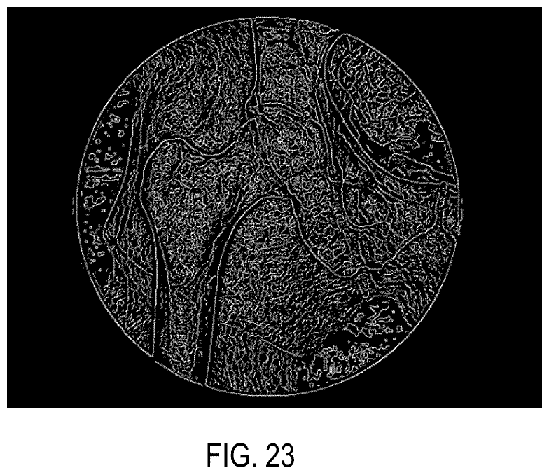

At step 2106, edge detection is performed on the two-dimensional image to identify the edges of the anatomy of interest in the image. For example, with respect to FIG. 22, edge detection may be used to identify edges of the femoral head, the femoral neck, the greater trochanter, the lesser trochanter, and/or any other portions of the femur. In an example, edge detection is performed on a sub-portion of the two-dimensional image defined based on one or more characteristics of the anatomy determined based on the feature detection of the machine learning model. For example, edge detection may be performed only on a predefined region surrounding the femoral head center estimate 2202 and/or femoral neck center estimate 2204. There are multiple ways to carry this edge detection step, including industry standard methods such as Sobel, Canny, and Scharr edge detection methods. FIG. 23 illustrates an example of the results of an edge detection algorithm for an upper portion of a femur, according to some embodiments.

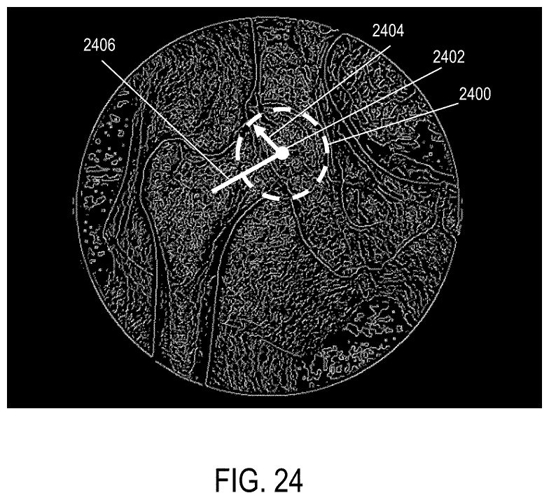

At step 2108, a final estimate of the one or more characteristics of the anatomical features is determined based on the edges detected in step 2106 and at least one of the initial estimates of one or more characteristics of the anatomical features from step 2104. For example, a final estimate of the center of the femoral head may be determined by first detecting the perimeter of the femoral head in the image. This can be done using a Hough transform, which looks for circles that match the edges of the femoral head. These circles may be limited in the range of the smallest and largest possible femoral heads. The Hough transform produces a list of possible answers and the best possible answer is selected. FIG. 24 illustrates an example of a circle 2400 from a Hough transform encircling the edges of the femoral head detected via edge detection.

Although the Hough transform is relatively fast, it may not be as accurate as desired since the femoral head may not be a perfect circle. An alternative approach includes using the initial estimate of the center of the femoral head and tracing along lines looking for edges between the minimum and maximum radii (which correlates to the smallest and largest possible femoral head). The minimum and maximum radii could be defined based on an initial estimate of the femoral head radius, such as determined based on the size of a bounding box for the femoral head. The point that has the strongest edge in each ray can be selected and checked to see if it aligns with other selected points to form a portion of a circle. Then another point is selected, and the process is repeated. This is done iteratively until the best point is found, using previous points as a guide for where to look next. Any other suitable technique can be used to locate the perimeter of the femoral head, including machine learned models trained on images of similar anatomy.

Once the femoral head is identified, a final estimate of the center of the femoral head in the x and y dimensions may be determined, as illustrated in FIG. 24 at 2402. Optionally, the radius of the femoral head may also be determined in step 2108 by measuring the distance from the center of the femoral head to the perimeter of the femoral head, as indicated at 2404 in FIG. 24.

Method 2100 continues with step 2110 in which the mid-line of the femoral neck is identified. The mid-line in the example of FIG. 24 is indicated at 2406. There are multiple ways to find the femoral neck. For example, a Box Sweep method can be used to find the femoral neck. A box is swept around the femoral head until the sides of that box line up with the edges of the femoral neck (as identified via edge detection). This is repeated for boxes of multiple sizes. The box that lines up with the strongest edges of the femoral neck can be chosen. The center of the box is then used to determine the mid-line of the femoral neck. To reduce the amount of searching required, the femoral neck center estimate 2204 may be used to define an aspect of the boxes, such by requiring boxes to be centered on the center estimate 2204. As one alternative, the mid-line may be determined as the lines connecting the femoral neck center estimate 2204 and the final femoral head center estimate from step 2108.

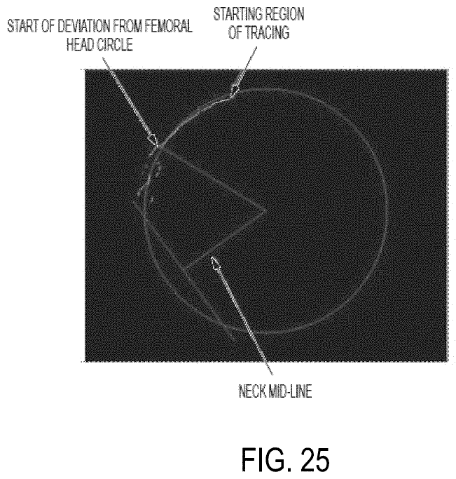

At step 2112, the location where the femoral head stops being round and the cam pathology starts is determined. For example, the strongest edges of the bone surface are traced (e.g., using the results of edge detection) until a deviation from the circle around the femoral head is found. As the region of interest is known, the tracing does not need to include the entire femoral head but rather just the region of interest. An examples is shown in FIG. 25. In identifying a deviation, a threshold level for the deviation can be used to ignore small deviations which may be a result of imperfections in edge detection rather than being the actual cam pathology. In one embodiment, the deviation threshold is a small percentage of the femoral head diameter, for example, 3-6% of the femoral head diameter, and more preferably 4% of the femoral head diameter. In another embodiment, the deviation threshold is a fixed value, for example, 0.5-2 mm, and more preferably 1 mm. In this embodiment, it is preferable to have calibrated the pixels of the image, so that the relative pixel size to the size of the anatomy is known.

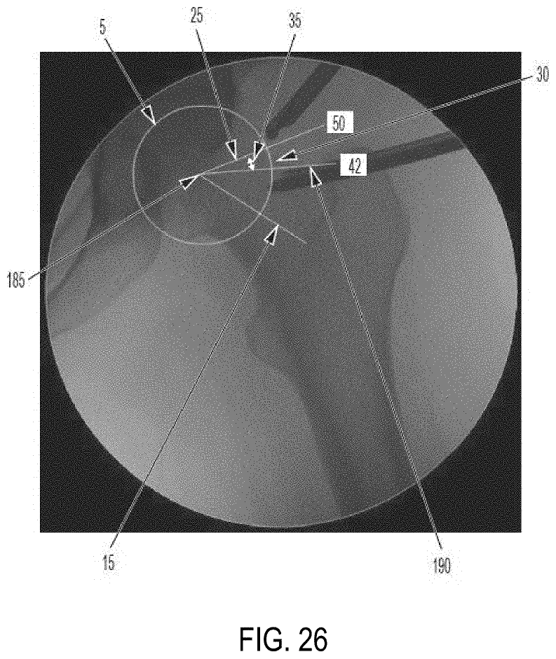

At step 2114, an Alpha Angle measurement is generated. As illustrated in the example of FIG. 26, the Alpha Angle 35 can be calculated as the angle between the mid-line 15 of the femoral neck, the center point 185 of the femoral head, and the location of the start of the cam pathology 30 at the femoral head/neck junction. In other words, the Alpha Angle is the angle measured between (i) the line 15 originating at the center of the femoral head and extending along the center of the femoral neck, and (ii) the line 25 originating at the center of the femoral head and passing through the location at the start of the cam pathology.

This Alpha Angle can be annotated onto the X-ray image, as shown in FIG. 26, along with circle 5 inscribing the femoral head and line 15 showing the center of the femoral neck, and this annotated X-ray image can be presented to the surgeon, such as on a display of computer visual guidance system 125 or other display. The surgeon may also find it useful to know the size of the cam pathology by way of the angle subtended between the Alpha Angle and a target Alpha Angle (i.e., the desired Alpha Angle), which can be provided via input from the surgeon or via another source. As such, the target Alpha Angle (line 190 in FIG. 26) can be included in a visualization displayed to a surgeon or other user, such as the visualization of FIG. 26, which can assist the surgeon in treating the pathology. The greater the difference between the Alpha Angle line 25 and the target Alpha Angle line 190, the larger the cam pathology and hence more bone removal is required.

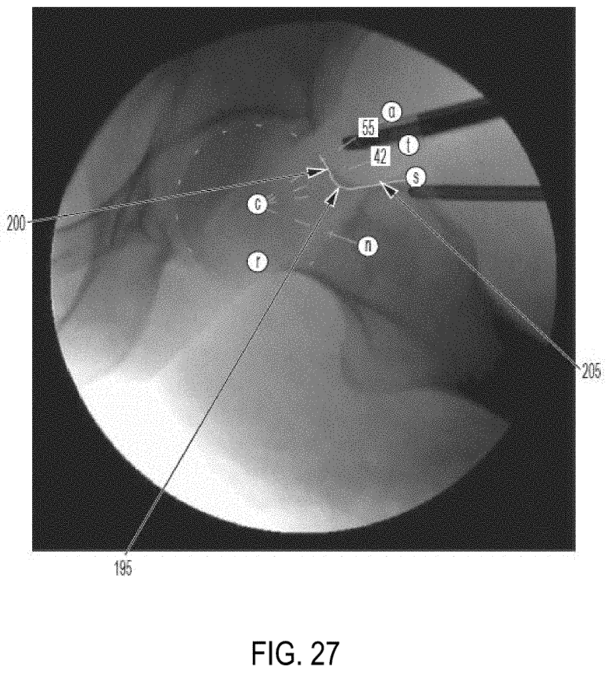

Optionally, method 2100 may also include automatically determining a resection curve based on the Alpha Angle. For example, with respect to the example of FIG. 27, the resection curve 195 comprises a first resection curve 200 adjacent to the femoral head, and a second resection curve 205 adjacent to the femoral neck. First resection curve 200 starts at the Alpha Angle Line 25 and ends at the target Alpha Angle line 190. Note that the first resection curve 200 can simply be the continuation of the circle of the femoral head. Second resection curve 205 starts at the end of first resection curve 200 (i.e., at the target Alpha Angle line 190) and extends down the neck. In some embodiments, second resection curve 205 may be concatenated to the end of first resection curve 200 so as to produce the overall resection curve 195. In some embodiments first resection curve 200 may comprise one or more curves and/or one or more lines, and/or may be referred to as a “resection curve portion” or simply a “portion.” In some embodiments, second resection curve 205 may comprise one or more curves and/or one or more lines, and/or may be referred to as a “resection curve portion” or simply a “portion.” In cases where the actual Alpha Angle is smaller than the target Alpha Angle, first resection curve 200 ends at the intersection of the actual Alpha Angle and the circle.

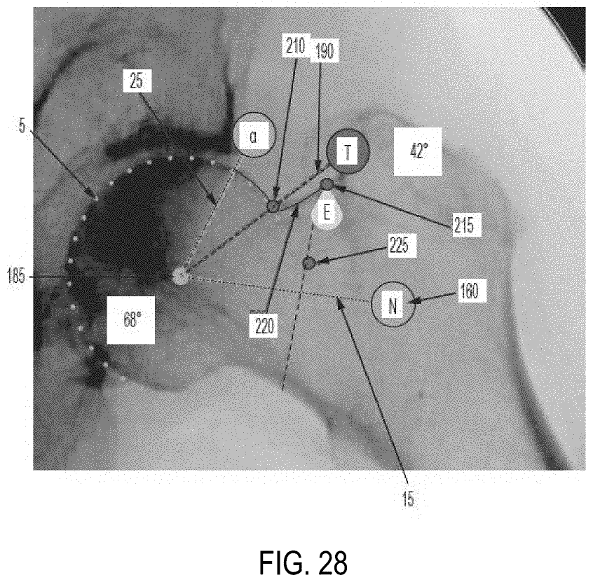

In some embodiments, second resection curve 205 is calculated as follows. First, and with reference to FIG. 28, the start point 210 and end point 215 of second resection curve 205 are found. As illustrated in FIG. 28, start point 210 is the point at which target Alpha Angle line 190 intersects the femoral head circle. Note that start point 210 is also the endpoint of first section curve 200. In some embodiments, end point 215 is found by determining the shortest distance between femoral neck hint 160 and the femoral neck boundary (edge): where this shortest line intersects the edge of the femoral neck defines end point 215. In some embodiments, end point 215 is on the edge of the femoral neck at its narrowest point. Then a spline 220 is generated, using start point 210, end point 215 and a control point 225 for spline 220. Note that spline 220 is second resection curve 205. The beginning of the second resection curve 205 can be tangent to the circle 5. Control point 225 for spline 220 may be generated in a variety of ways. By way of example but not limitation, control point 225 may be obtained by studying a set of “normal” patient anatomies and determining an appropriate control point for a given start point 210 and a given endpoint 215 in order to provide a spline approximating a normal anatomy. In some embodiments, control point 225 may be obtained by polling a group of experts to determine an appropriate control point for a given start point 210 and a given endpoint 215 in order to provide a spline approximating a normal anatomy. In some embodiments, control point 225 may be obtained on a line extending tangent to the end of first resection curve 200 a distance that is proportional to the radius of the femoral head. In some embodiments, after start point 210, end point 215 and control point 225 have been determined, spline 220 (i.e., second resection curve 205) is generated and displayed with the X-ray image. In some embodiments, spline 220 is a Bezier curve.

### Generating Head-Neck Offset According to Method 1900

An alternative measurement to the Alpha Angle measurement that can be used for diagnosing and treating a cam pathology is the head-neck offset. Accordingly, method 1900 may be used for generating head-neck offset measurements and resection curves generated based on those measurements as an alternative to Alpha Angle based measurements and resection curve generation of FIG. 21. In an example, a user may switch between these two methods based on the user's preference.

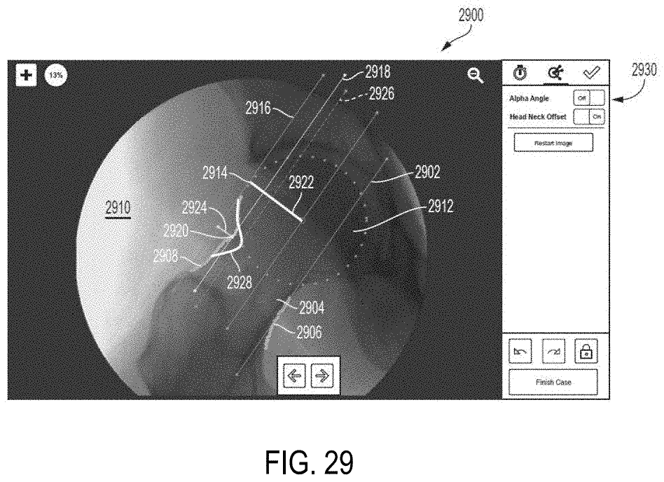

FIG. 29 is an example of a graphical user interface 2900 that can be generated, such as by computer visual guidance system 125, for providing a user with head-neck offset measurements and a resection curve based on those measurements, according to an example of method 1900. With reference to FIG. 29, the system may be configured to determine the head-neck offset and resection curve based on that head-neck offset, as discussed further below.

The process for determining the head-neck offset may include detecting the femoral head and/or femoral neck, according to step 1904 of method 1900. Then, one or more characteristics of the head and/or neck can be determined in step 1906, including, for example, the center and/or perimeter of the femoral head and/or the center and/or perimeter of the femoral neck. These characteristics can be used directly or as initial estimates that are used to determine final estimates of the characteristics. For example, an initial estimate of the center of the femoral head can be used to generate a final estimate of the best fit head circle 2901 of FIG. 29. Next, a line 2902 that extends along the lower edge 2906 of the femoral neck is estimated, such as by locating the lower edge 2906 in the X-ray image 2910 using any suitable edge detection method and creating a line that passes through a majority of points of the lower edge 2906. Next, two lines that are parallel to line 2902 are determined. A femoral head line 2916 that is parallel to the centerline 2902 and is tangent to the superior side of the femoral head 2912 (the point of tangency is indicated by reference numeral 2914) is determined, and a femoral neck line 2918 that is parallel to centerline 2902 and is tangent to a most-recessed point 2920 of the superior side of the femoral neck 2904 in the image. A neck measurement 2924 is taken from the femoral neck line 2918 to the femoral head line 2916, and the ratio of the neck measurement 2924 to the radius 2922 of the femoral head is the head-neck offset.

A head-neck offset that is too small may be associated with cam pathology and treatment of the cam pathology based on the head-neck offset measurement may include resecting the bone until the head-neck offset (as determined according to the steps above) is at or above a target head-neck offset. The target head-neck offset can be defined by a user or, such as based on measurements in non-pathologic joints (for example, from patient studies reported in literature). In some embodiments, a predefined target value may be adjustable by a user. An example of a target head-neck offset is 17%, which means that head-neck offsets below this number may be associated with a cam pathology.

In FIG. 29, the target head neck offset is shown by the dashed line 2926, which indicates where the most-recessed portion of the neck should be (i.e., wherein the femoral neck line 2918 should be) to achieve the target head-neck morphology. The measured head-neck offset in the illustrated example is 13%, as indicated in the top left of the user interface 2900. Since this value is below the target value (as represented by the target line 2926 being below the femoral neck line 2918) in this example, the head-neck area of the femur should be resected to achieve the target morphology.

The computer visual guidance system 125 may generate a resection curve 2928 based on the target head-neck offset to indicate how the bone should be resected to achieve the target head-neck offset. Generally, according to various embodiments, the resection curve is generated by following the perimeter of the femoral head down to the target line 2926 and then curving back up to align with the edge 2908 of the neck 2904.

According to various embodiments, the graphical user interface 2900 can include one or more user selectors 2930 for selecting between Alpha Angle based resection curve generation and head-neck ratio based resection curve generation. FIG. 29 is merely exemplary of various embodiments of graphical user interface 2900 and it should be understood that any of the visual indications overlaid on the X-ray image in FIG. 29 may be included or excluded, according to various embodiments. Optionally, the features overlaid on the X-ray image 2910 can be user-adjusted. For example, a user may select the centerline 2902 and drag the centerline 2902 (such as via a touchscreen or mouse) to a location that the user determines to be closer to the center of the neck or the user could select and drag the target head-neck offset line 2918 to increase or decrease the depth of the resection curve.

### Generating Center Edge Angle According to Method 1900

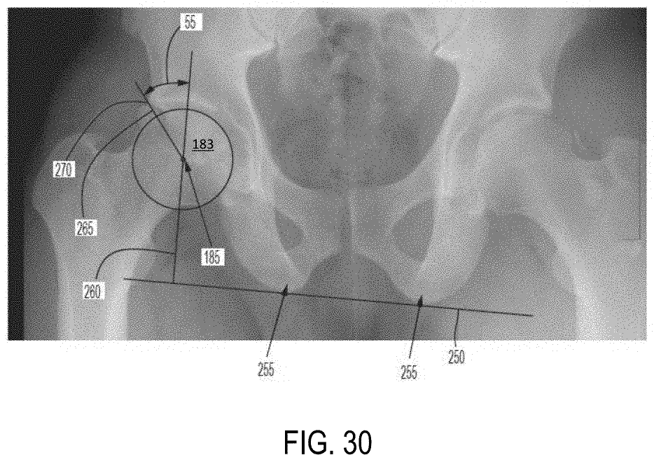

Method 1900 can additionally or alternatively be used to determine a Center Edge Angle, such as for guiding a surgeon in treating a pincer-type cam pathology. Referring to the example illustrated in FIG. 30, the Center Edge Angle 55 can be determined according to step 1908 of method 1900 using a perpendicular 260 to a transverse pelvic axis 250 and a lateral acetabular edge line 265. These components are determined from characteristics of anatomical features detected by a machine learning model, according to steps 1904 and 1906. The anatomical features detected by the machine learning model that may be used for determining the Center Edge Angle can be and/or include the inferior apexes 255 of the ischium bones, one or more of the femoral heads 183, and the lateral edge 270 of the acetabular rim 271. Aspects of bounding boxes generated via the machine learning model according to step 1904 may be used directly as characteristics of these features or may be used for determining those characteristics, according to step 1906. For example, the centers of bounding boxes for the femoral heads may be used as the centers of the femoral head or used as initial estimates of those centers, as discussed above. Similarly, the centers or edges of bounding boxes for the inferior apexes 255 and/or the lateral edge 270 of the acetabular rim 271 may be used directly as locations for the features or may be used to derive the locations of the features in similar fashion to the derivation of the centers of the femoral heads from their bounding box centers, as discussed above.

At step 1908, the transverse pelvic axis 250 is determined as a line that extends along the inferior apexes 255 of the ischium bones or that extends through the centers of both femoral heads. Once the transverse pelvic axis 250 has been generating, a perpendicular 260 to the transverse pelvic axis 250 that extends through the center 185 of the femoral head is determined, such as by extending a line from the center 185 of the femoral head that is 90 degrees from the transverse pelvic axis 250. Next, the lateral acetabular edge line 265 is determined by extending a line from the lateral edge 270 of the acetabular rim 271 to the center 185 of the femoral head. The Center Edge Angle 55 (i.e., the angle between the perpendicular 260 and the lateral acetabular edge line 265) is calculated, e.g., by measuring the angle formed between the portion of the perpendicular 260 on the superior side of the femoral head and the lateral acetabular edge line 265. The Center Edge Angle measurement generated according to step 1908 can be provided in a visualization displayed to the user.

### Generating Anatomy Orientation According to Method 1900

Method 1900 can additionally or alternatively be used to determine at least one orientation of anatomy of interest of a patient from two-dimensional imaging. For example, an orientation of a femur and/or pelvis may be determined from two-dimensional imaging capturing the femur and/or pelvis. A plurality of features of the femur and/or pelvis can be detected by a machine learning model, according to step 1904, the locations the features can be determined in step 1906, and those locations relative to one another can be used to generate an orientation of the femur and/or pelvis in step 1908. This could be done, for example, using a regression machine learning model trained to generate orientation(s) based on relative locations of the detected features. As used herein, “orientation” refers to an angle of an object about any single axis relative to a reference point and does not refer to the entire three-dimensional characterization of the pose of an object. Thus, a single object may have multiple orientations, each being relative to a different axis of rotation.

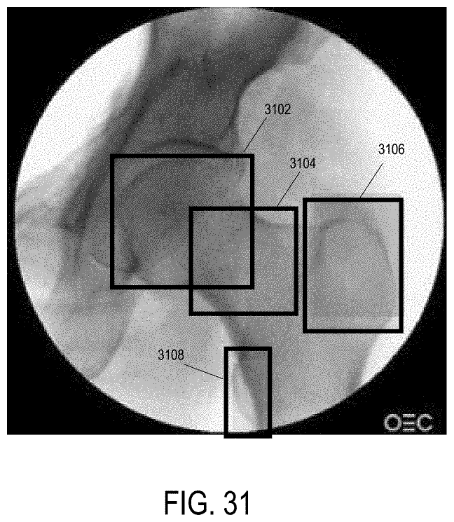

For example, with reference to FIGS. 1A-D, an orientation of a femur generated according to method 1900 could include one or more of a degree of flexion or extension from a neutral position, a degree of abduction or adduction from a neutral position, and/or a degree of medial or lateral rotation from a neutral position. FIG. 31 illustrates an example of features of a femur detected by a machine learning model, according to step 1904, that can be used to determine an orientation of the femur. In the illustrated embodiment, the machine learning model detected the femoral head 3102, the femoral neck 3104, the greater trochanter 3106, and lesser trochanter 3108. The relative locations of the features (e.g., determined using the centers of their respective bounding boxes) can be used to determine one or more degrees of orientation of the femur. For example, a more vertical alignment of the greater and lesser trochanter in an anterior-posterior X-ray image could correspond to a greater degree of abduction of the femur.

An orientation of a pelvis generated according to method 1900 could include a degree of anterior or posterior pelvic tilt. This measurement could be generated based on a shape of the obturator foramen, which can correspond to a degree of tilt of the pelvis when viewed in an anterior-posterior view. For example, after detection of the obturator foramen in the image, a regression machine learning model may be used to determine the pelvic tilt from the shape of the obturator foramen. Orientations of a pelvis generated according to method 1900 can additionally or alternatively include pelvic incidence, pelvic obliquity, and hip flexion/extension. In some examples, the orientation of a pelvis generated according to method 1900 may based on the relative location of the acetabular edges (e.g., the superior and/or inferior edges).

The orientation(s) of the femur and/or pelvis may be displayed to a user. Additionally or alternatively, the orientation(s) may be used for other purposes. For example, a neutral pelvic tilt (in absolute terms or relative to the imager) may be important in generating other measurements of the hip, and the pelvis orientation can be used to determine whether the degree of tilt of the pelvis is too great to produce accurate measurements. The tilt of the pelvis relative to the imaging perspective may be compared to a predetermined threshold, and if the tilt is greater than the threshold, a warning may be provided to a user informing them that the pelvis is over-tilted relative to the imaging perspective and that new imaging is needed in which the pelvis is not over-tilted. The user could reposition the patient or could reposition the imager and capture new imaging. Additionally or alternatively, the degree of tilt could be factored into the determination of one or more measurements.

Additionally or alternatively, orientation(s) of a femur and/or pelvis can be used to align a three-dimensional model of the anatomy to the two-dimensional imaging. The three-dimensional model may have been generated from three-dimensional imaging of the patient (such as MM or CT scans) and may be used to provide the user with additional information and/or three-dimensional visualizations (such as three-dimensional visualization of the anatomy of interest). This three-dimensional information can be overlaid on or otherwise combine with the two-dimensional imaging by first determining the orientation of the anatomy in the imaging. Orientation(s) of the anatomy of interest in the two-dimensional imaging, determined according to method 1900, can be used directly to align the three-dimensional model to the two-dimensional imaging or can be used to inform an algorithm that determines that alignment.

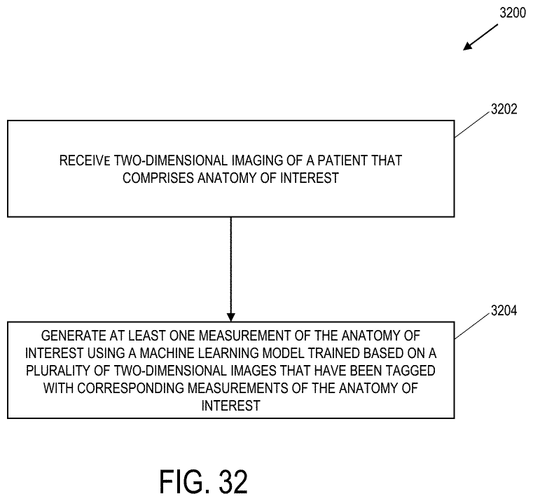

As described above, a machine learning model can be used to detect anatomical features in two-dimensional imaging, and characteristics of those detected features can be used to generate one or more measurements of the anatomy of interest. Alternatively, a machine learning model can be used to generate the measurements themselves. FIG. 32 is a block diagram of a method 3200 that uses one or more machine learning models to generating at least one measurement of anatomy of interest from two-dimensional imaging. Method 3200 is performed by a computing system, such as visual guidance system 125 of FIG. 18.

At step 3202, two-dimensional imaging, such as X-ray imaging, of a patient that comprises the anatomy of interest is received by the computing system. At step 3204, at least one measurement of the anatomy of interest is generating using at least one machine learning model trained based on a plurality of two-dimensional images that have been tagged with corresponding measurements of the anatomy of interest. The one or more machine learning models can be trained to generate any desired anatomical measurement, including, for example, Alpha Angle, Center Edge Angle, a head-neck offset, Tönnis angle (also referred to as acetabular inclination and acetabular index), acetabular version, femoral torsion, femoral version, acetabular coverage, orientation of a femur and/or pelvis, and femoral neck shaft angle.

In some examples, the trained machine learning models may be re-trained using log images, which are images captured and automatically or manually logged/recorded during surgical procedures and subsequently made available for re-training. The log images may include images with one or more features making the image particularly difficult for the machine learning model to process. For instance, the log images may include one or more tools in the image that may partially block the anatomy of interest, images of slipped capital femoral epiphysis (SCFE), or oval-shaped (or otherwise uncommonly shaped) femoral heads, or any combination of these features.

The plurality of two-dimensional images used to train the machine learning model may be images captured during medical procedures and/or in cadaver labs. In some examples, the plurality of two-dimensional training images used to train the machine learning model may include randomly placed tools within the image. The randomly placed tools may simulate tools left in the image frame for images captured during a medical procedure. The training images could additionally or alternatively be pseudo two-dimensional images generated from at least one three-dimensional imaging data set. The three-dimensional imaging data set can be, for example, CT or MRI scans of one or more subjects. The three-dimensional imaging data is used to generate pseudo two-dimensional images capturing the anatomy of interest from different perspectives by flattening the three-dimensional imaging data according to known methods. The pseudo two-dimensional images can be altered to make them look more like actual two-dimensional imaging images. For example, a generative adversarial network (GAN) or a style transfer can be used to generate two-dimensional images that are more similar to actual two-dimensional imaging modality images. The more realistic pseudo two-dimensional images can be altered to reduce image quality, again, to make the pseudo two-dimensional images more like actual two-dimensional images. This step can include increasing or decreasing contrast, adding noise to the data, adding artifacts to the images, such as to mimic a tool being in the field of view. By using or including pseudo two-dimensional imaging, the training data set can be greatly expanded relative to a training data set limited to real two-dimensional images.

Whether natural or pseudo two-dimensional images, the images are then tagged with the desired measurements of the anatomy of interest. A machine learning model trained on this data can provide the measurement(s) from two-dimensional imaging. The machine learning model may include multiple analysis steps, such as one or more object detection stages to detect anatomical features that are followed by one or more regression stages used to generate the desired measurement(s) from the detected anatomical features. For example, with respect to the tilt of the pelvis, an object detection stage of a machine learning model can detect the obturator foramen in an X-ray image of the pelvis, and the shape of the obturator foramen can be analyzed by a regression stage of the machine learning model to generate the pelvic tilt measurement. The measurements may be displayed to a user in a visualization to assist in treating a hip joint pathology or for informing further analysis, as discussed above.

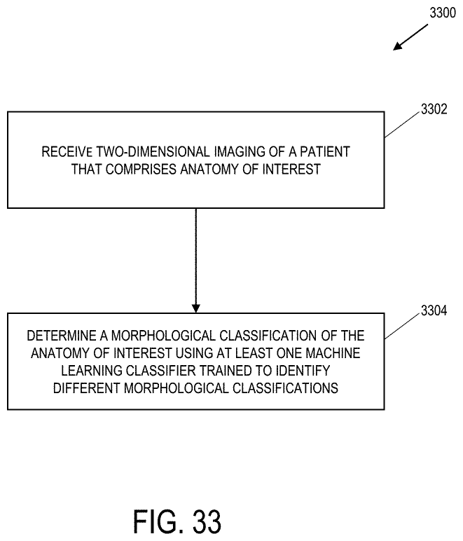

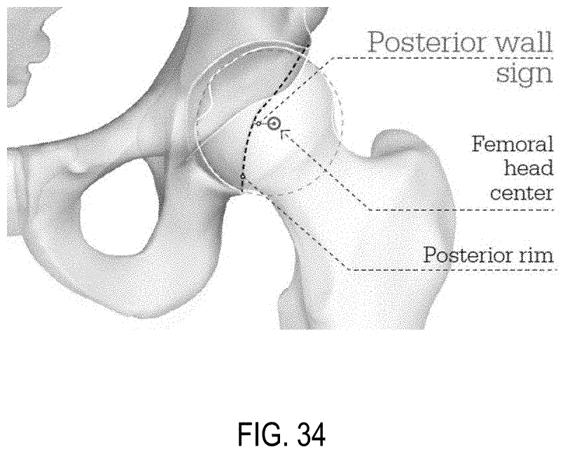

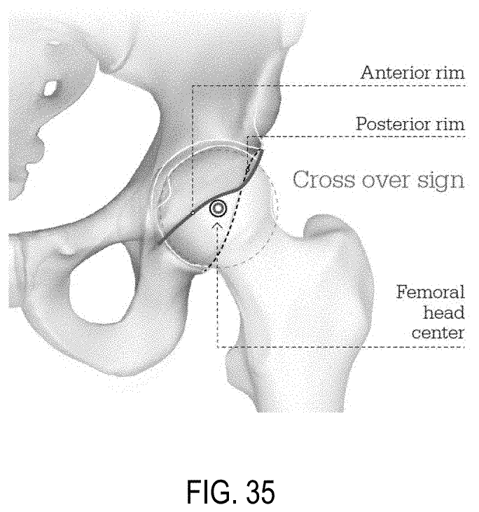

Machine learning models may additionally or alternatively be used to classify anatomical morphology in two-dimensional imaging. FIG. 33 is a diagram of a method 3300 for determining a morphological classification of anatomy of interest. At step 3302, two-dimensional imaging of a patient that comprises the anatomy of interest is received by a computing system, such as visual guidance system 125 of FIG. 18. At step 3304, a morphological classification of the anatomy of interest using at least one machine learning classifier trained to identify different morphological classifications. FIGS. 34-35 illustrate different morphological classifications for a hip joint that may be identified according to method 3300. FIG. 34 illustrates a posterior wall sign morphology in which the outline of the posterior acetabular wall projects over the center of the femoral head. FIG. 35 illustrates a crossover sign in which a line drawn along the anterior rim of the pelvis crosses over a line drawn along the posterior rim. Another example of a hip joint morphology classification is an ischial spine sign in which a triangular projection of the ischial spine is visible medially to the pelvic inlet. Other examples of hip joint morphological classifications include acetabular cup depth, Shenton's line, and teardrop sign. One or more machine learning models may be trained to detect these and other joint morphology classifications using training images labeled with the joint morphology classifications.

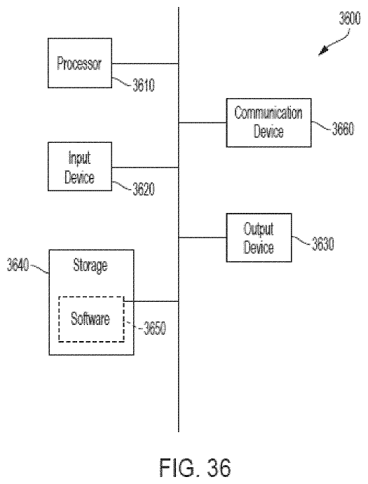

FIG. 36 illustrates an example of a computing system 3600 that can be used for performing one or more steps of the methods described herein, including one or more steps of method 1900 of FIG. 19, method 2100 of FIG. 21, method 3200 of FIG. 32, and method 3300 of FIG. 33. Computing system 3600 could be used, for example, visual guidance system 125 of FIG. 18. System 3600 can be a computer connected to a network, such as one or more networks of hospital, including a local area network within a room of a medical facility and a network linking different portions of the medical facility. System 3600 can be a client or a server. As shown in FIG. 36, system 3600 can be any suitable type of processor-based system, such as a personal computer, workstation, server, handheld computing device (portable electronic device) such as a phone or tablet, or dedicated device. The system 3600 can include, for example, one or more of input device 3620, output device 3630, one or more processors 3610, storage 3640, and communication device 3660. Input device 3620 and output device 3630 can generally correspond to those described above and can either be connectable or integrated with the computer.

Input device 3620 can be any suitable device that provides input, such as a touch screen, keyboard or keypad, mouse, gesture recognition component of a virtual/augmented reality system, or voice-recognition device. Output device 3630 can be or include any suitable device that provides output, such as a display, touch screen, haptics device, virtual/augmented reality display, or speaker.

Storage 3640 can be any suitable device that provides storage, such as an electrical, magnetic, or optical memory including a RAM, cache, hard drive, removable storage disk, or other non-transitory computer readable medium. Communication device 3660 can include any suitable device capable of transmitting and receiving signals over a network, such as a network interface chip or device. The components of the computing system 3600 can be connected in any suitable manner, such as via a physical bus or wirelessly.

Processor(s) 3610 can be any suitable processor or combination of processors, including any of, or any combination of, a central processing unit (CPU), graphics processing unit (GPU), field programmable gate array (FPGA), and application-specific integrated circuit (ASIC). Software 3650, which can be stored in storage 3640 and executed by one or more processors 3610, can include, for example, the programming that embodies the functionality or portions of the functionality of the present disclosure (e.g., as embodied in the devices as described above). For example, software 3650 can include one or more programs for execution by one or more processor(s) 3610 for performing one or more of the steps of method 1900 of FIG. 19, method 2100 of FIG. 21, method 3200 of FIG. 32, and method 3300 of FIG. 33.

Software 3650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 3640, that can contain or store programming for use by or in connection with an instruction execution system, apparatus, or device.

Software 3650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport computer readable medium can include, but is not limited to, an electronic, magnetic, optical, electromagnetic, or infrared wired or wireless propagation medium.

System 3600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 3600 can implement any operating system suitable for operating on the network. Software 3650 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method of generating a measurement of anatomy of interest from two-dimensional imaging, comprising:
receiving two-dimensional imaging associated with anatomy of interest;
detecting a plurality of anatomical features of the anatomy of interest in the two-dimensional imaging using at least one machine learning model;
determining characteristics of the plurality of anatomical features based on the detection of the plurality of anatomical features; and
generating at least one measurement of the anatomy of interest based on at least some of the characteristics of the plurality of anatomical features.

2. The method of claim 1, wherein determining the characteristics of the plurality of anatomical features comprises determining an initial estimate of a characteristic of a first anatomical feature based on the detection of the plurality of anatomical features and determining a final estimate of the characteristic of the first anatomical feature based on the first estimate.

3. The method of claim 2, wherein the initial estimate of the characteristic of the first anatomical feature comprises an estimate of at least one of a location and a size of the first anatomical feature, and determining the final estimate comprises searching for a perimeter of the first anatomical feature based on the estimate of at least one of the location and the size of the first anatomical feature.

4. The method of claim 1, wherein the plurality of anatomical features comprises a head and neck of the femur and the characteristics comprise a location of mid-line of the neck.

5. The method of claim 1, wherein the at least one measurement comprises an Alpha Angle generated based on the location of the mid-line.

6. The method of claim 5, further comprising automatically generating a resection curve based on the Alpha Angle.

7. The method of claim 1, wherein the plurality of anatomical features detected comprises a plurality of features of a femur and the at least one measurement comprises an orientation of the femur relative to a predefined femur orientation.

8. The method of claim 7, further comprising determining an alignment of a three-dimensional model of the femur with the two-dimensional imaging based on the orientation of the femur.

9. The method of claim 7, further comprising comparing the orientation to a predefined orientation threshold and, in response to determining that the orientation is beyond the predefined orientation threshold, notifying the user.

10. The method of claim 1, wherein the at least one machine learning model generates a plurality of scored bounding boxes for the plurality of anatomical features and the characteristics of the plurality of anatomical features are determined based on bounding boxes that have scores that are above a predetermined threshold.

11. The method of claim 1, further comprising displaying a visual guidance associated with the anatomy of interest based on the at least one measurement.

12. The method of claim 11, wherein the visual guidance provides guidance for bone treatment.

13. The method of claim 1, wherein the plurality of anatomical features detected comprises a plurality of features of a pelvis and the at least one measurement comprises an orientation of the pelvis relative to a predefined pelvis orientation.

14. The method of claim 13, further comprising comparing the orientation to a predefined orientation threshold and, in response to determining that the orientation is beyond the predefined orientation threshold, notifying the user.

15. The method of claim 1, wherein the at least one measurement of the anatomy of interest is generated using a regression machine learning model.

16. A method of generating a measurement of anatomy of interest from two-dimensional imaging, comprising:
receiving two-dimensional imaging of a patient that comprises the anatomy of interest; and
generating at least one measurement of the anatomy of interest using a machine learning model trained based on a plurality of two-dimensional images that have been tagged with corresponding measurements of the anatomy of interest.

17. The method of claim 16, wherein the plurality of two-dimensional images comprises a plurality of pseudo two-dimensional images generated from at least one three-dimensional imaging data set.

18. The method of claim 16, wherein the anatomy of interest comprises a femur or a pelvis and the measurement comprises an orientation of the femur or pelvis.

19. The method of claim 16, wherein the anatomy of interest is a hip joint and the at least one measurement comprises Alpha Angle, head-neck offset, Center Edge Angle, Tönnis angle, acetabular version, femoral version, acetabular coverage, or femoral neck shaft angle.

20. The method of claim 16, further comprising displaying a visual guidance associated with the anatomy of interest based on the at least one measurement.

21. The method of claim 20, wherein the visual guidance provides guidance for bone treatment.

22. The method of claim 16, wherein the at least one measurement comprises at least one pelvic orientation, and generating the at least one measurement comprises detecting an obturator foramen and determining the at least one pelvic orientation based on the obturator foramen.

23. The method of claim 22, wherein determining the at least one measurement comprises analyzing the obturator foramen using a regression machine learning model.

24. A method for determining a morphological classification of anatomy of interest, comprising:
receiving two-dimensional imaging of a patient that comprises the anatomy of interest; and
determining the morphological classification of the anatomy of interest using at least one machine learning classifier trained to identify different morphological classifications.

25. The method of claim 24, wherein the anatomy of interest is a hip and the morphological classification comprises a posterior wall sign, a crossover sign, an ischial spine sign, an acetabular cup depth, a Shenton's line, and a teardrop sign.

26. A system for generating a measurement of anatomy of interest from two-dimensional imaging, the system comprising one or more processors, memory, and one or more programs stored in the memory for execution by the one or more processors for causing the system to:
receive two-dimensional imaging associated with anatomy of interest;
detect a plurality of anatomical features of the anatomy of interest in the two-dimensional imaging using at least one machine learning model;
determine characteristics of the plurality of anatomical features based on the detection of the plurality of anatomical features; and
generate at least one measurement of the anatomy of interest based on at least some of the characteristics of the plurality of anatomical features.

27. A system for generating a measurement of anatomy of interest from two-dimensional imaging, the system comprising one or more processors, memory, and one or more programs stored in the memory for execution by the one or more processors for causing the system to:
receive two-dimensional imaging of a patient that comprises the anatomy of interest; and
generate at least one measurement of the anatomy of interest using a machine learning model trained based on a plurality of two-dimensional images that have been tagged with corresponding measurements of the anatomy of interest.

28. A system for determining a morphological classification of anatomy of interest, the system comprising one or more processors, memory, and one or more programs stored in the memory for execution by the one or more processors for causing the system to:
receive two-dimensional imaging of a patient that comprises the anatomy of interest; and
determine the morphological classification of the anatomy of interest using at least one machine learning classifier trained to identify different morphological classifications.
